(19) United States
(12) Patent Application Publication
(10) Pub. No.: US 2018/0011983 A1
(43) Pub. Date: Jan. 11, 2018

(54) METHOD AND SYSTEM FOR MANAGING MEDICAL DATA

(71) Applicant: THINK SURGICAL, INC., Fremont, CA (US)

(72) Inventors: Joel Zuhars, Fremont, CA (US); Saleh TABANDEH, Fremont, CA (US)

(21) Appl. No.: 15/548,121

(22) PCT Filed: Feb. 1, 2016
(86) PCT No.: PCT/US2016/015909

§ 371 Date: Aug. 2, 2017

Related U.S. Application Data
(60) Provisional application No. 62/111,016, filed on Feb. 2, 2015.

(51) Int. Cl. G06F 19/00; G06Q 50/22; H04B 10/116

(52) U.S. Cl. CPC G06F 19/3406; H04B 10/116; G06Q 50/22; A61B 34/10; A61B 34/30; A61B 2034/2055

(57) ABSTRACT

A method to securely transfer data to aid in a medical procedure or to a data management system to be accessed in a medical care facility is provided that includes a set of data from a data storage system being downloaded via a network to a visible light communication (VLC) enabled device. The set of data from the VLC enabled device is then transferred to a VLC enabled device located in an operating room (OR) via point to point VLC or uploaded to a data management system. The transferred set of data is then used to aid in a medical procedure or be accessed by a medical care facility. Software executables are similarly transferred and run in an OR context.

### RELATED APPLICATIONS

This application claims priority benefit of U.S. Provisional Application Ser. No. 62/111,016 filed 2 Feb. 2015; the contents of which are hereby incorporated by reference.

### FIELD OF THE INVENTION

The present invention relates generally to the field of medical data management, and more specifically to a new and useful method for using point to point visible light communication for the safe and secure

### BACKGROUND OF THE INVENTION

Medical care facilities maintain a substantial amount of data including patient records, patient data, scheduling, equipment lists, and inventory. The transfer and management of the data is critical to ensure the quality of care to patients, efficiency of medical practitioners, and to effectively manage a medical care facility. The Health Insurance Portability and Accountability Act (HIPPA) stipulates privacy rules that further require that personal health information is maintained and transferred according to strict guidelines to ensure the information is protected.

In a fast paced hospital setting, surgeons and healthcare workers traverse multiple rooms and operating suites to perform different procedures. In particular, surgical procedures require surgical plan data that must be properly and securely stored, managed, and quickly transferred and associated with the correct patient, any required medical personnel, the correct operating room, and the time of the procedure. The surgical plan data may further include patient information, the surgical plan, medical images, necessary medical devices or instruments, and any data required to operate a medical device. Generally, this information is stored on paper or in electronic files whereby the need to locate and/or transfer the specific data can be tedious and time consuming especially in cases of high patient volume or an emergency.

Additionally, an operating room contains several different types of surgical equipment such as diagnostic tools, respiratory and cardiac support, patient monitors, and computer assisted medical devices. All of this equipment requires maintenance and calibration to ensure that the pieces of equipment are functioning properly. Often, the calibration and any required maintenance is logged on paper or in an electronic file, which requires the users to manually check and keep track of what equipment needs any particular attention. If any of the periodic maintenance of the equipment is missed, then the equipment may be operating outside of defined ranges or fail altogether.

More specifically, certain medical devices, for example robotic surgical equipment, such as the ROBODOC™ Surgical System (THINK Surgical, Fremont, Calif.), require a pre-operative surgical plan data file to provide instructions to the computer assisted device to accurately mill the femoral bone cavity of a patient undergoing total hip arthroplasty. Generally the pre-operative data files contain large amounts of data, and the data files are stored on compact discs (CDs) or universal serial bus (USB) drives, which are additional physical files that need to be maintained, managed and transferred safely and securely. Furthermore, physical files may also be prone to damage or corruption.

Thus, there is a need for an effective, secure and fast and efficient way to manage medical data in a medical care facility. There is an additional need to transfer large amounts of surgical data without the use of storing and transferring the data on a physical medium.

### SUMMARY OF THE INVENTION

A method to securely transfer data to aid in a medical procedure is provided that includes a set of data from a data storage system being downloaded via a network to a visible light communication (VLC) enabled mobile device. The set of data from the VLC enabled mobile device is then transferred to a VLC enabled device located in an operating room (OR) via point to point VLC. The transferred set of data is then used to aid in a medical procedure.

A method to securely transfer operating room (OR) data to a data management system to be accessed in a medical care facility is also provided that includes data from the OR being transferred to a visible light communication (VLC) enabled device via point to point VLC. The data from the VLC enabled device being uploaded to a data management system. The uploaded data being stored in the data management system to be accessed by a medical care facility.

A method to transfer software executables on operating room (OR) equipment to perform a medical procedure is provided that includes software executable being downloaded via a network to a visible light communication (VLC) enabled device. The software executable is transferred from the VLC enabled mobile device to the OR equipment via point to point VLC. The software executable is then run on the OR equipment to perform a medical procedure.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 is a flowchart of the method for surgical plan data transfer to an operating room (OR) in accordance with embodiments of the invention;

FIG. 2 is a schematic of the transfer of surgical plan data within the OR in accordance with embodiments of the invention;

FIG. 3 is a schematic of the transfer of surgical plan data as part of a larger cloud based data management and computational system implemented within the OR in accordance with embodiments of the invention; and

FIG. 4 is a schematic diagram illustrating an overall view of communication devices, computing devices, and mediums for implementing embodiments of the invention;

FIG. 5 is an illustrative depiction of a VLC enabled mobile device in accordance with embodiments of the invention.

### DETAILED DESCRIPTION OF THE INVENTION

The invention disclosed herein describes a system and method for data transfer in a medical care facility, but more particularly to the secure transfer of data in and out of an operating room.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-4, 3-4, and 1-4.

The invention described herein refers to the use of visible light communication (VLC). Visible light communication (VLC) is a data communications medium using visible light between 400 and 800 THz (10¹² Hz, 780-375 nm). It should be appreciated that point to point VLC has advantages for connectivity and security that are particularly important within the operating room (OR), including unambiguous device to device linkage, low latency of data transmission, and non-interference with RF communication devices used in the location. Additionally, with point to point VLC, the data is targeted between specific transmitters and receivers for added security and specificity that distinguishes itself from the light fidelity (LiFi) or light networking concepts.

Generally in VLC the data for transmission is first pre-processed using various signal processing operations such as error-correction code encoding, modulation, multiplexing, interleaving, and/or fast Fourier transforms (FFT). A light emitting element drive circuit then actuates a light emitting diode (LED) with the desired signal. A light detecting sensor such as a photodiode, CCD or CMOS camera detects the signal, which is then post-processed to read the signal. It should be appreciated that data transfer rates have been achieved greater than 1 Gb/s.

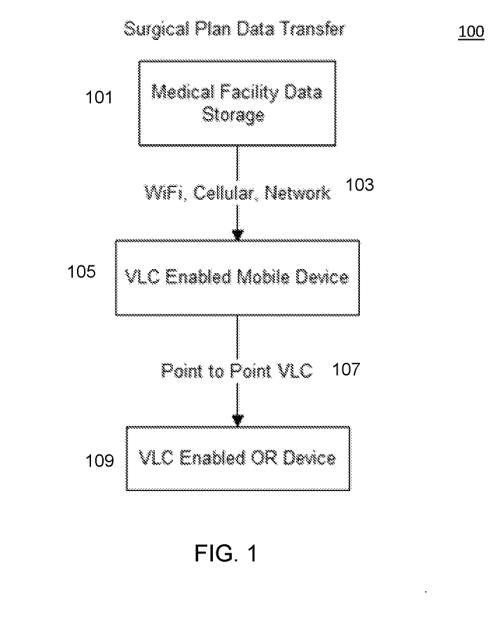

Referring now to the figures, FIG. 1 illustrates a flowchart 100 of an embodiment of an inventive method for securely transferring data to an operating room. A medical facility data storage system 101 contains a plurality of data for a medical care facility. The A medical facility data storage system 101 may be any system capable of storing data and wirelessly connecting to external devices such as a cloud based storage system, network, or server based system. The stored data may include patient information with their diagnosis, medical history, facility identification code, surgical procedure, operational site, pre-operative images, pre-operative plans, any special considerations such as previous incisions or operations, surgical plans from previous operations, any data or information necessary to operate specific medical devices, and/or software executables that can be run to use equipment for an arbitrary surgical procedure, with or without the use of an associated surgical plan. The A medical facility data storage system 101 may also include data linked with hospital case management information such as the operating times and locations, the required medical staff, equipment and/or supplies to execute a given procedure or surgery, the operational status of equipment such as battery life, calibration information, maintenance schedule, and maintenance plans.

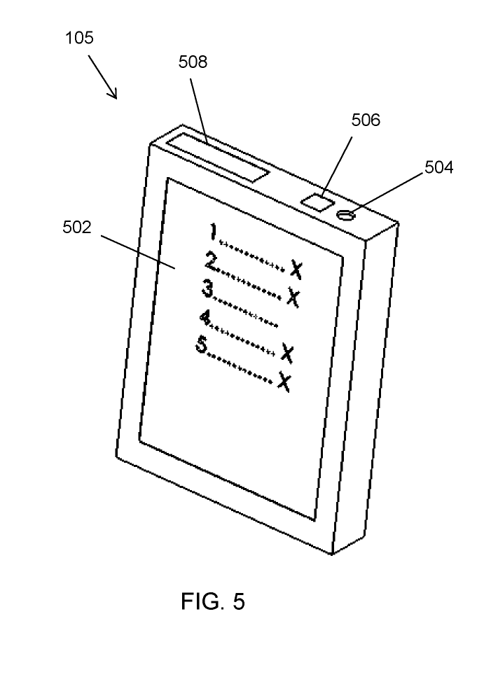

A visible light communication (VLC) enabled mobile device 105 is used to download surgical plan data from the data storage system 101 by wireless communications 103 such as WiFi, a cellular network, or a wireless local area networks such as a medical care facility network. The VLC mobile device 105 may be for example a smart phone, tablet, a dongle or an external device capable of wirelessly downloading data from the data storage system 101. In one embodiment, the mobile device 105 may be equipped with VLC capabilities by, for example, integrating or plugging in an external VLC module that integrates with the mobile device 105 (e.g. through an auxiliary input). In another embodiment the mobile device 105 has VLC capabilities already integrated into the device. In a specific embodiment, the VLC enabled mobile device 105 has bidirectional data transfer capabilities so data can be sent and received via VLC. An example of a VLC enabled mobile device is shown in FIG. 5. The VLC enabled mobile device 105 includes a display 502, an LED 504 for transmitting data, a photodiode 506 for receiving data, and an optional bar code scanner 508. A camera (not shown) integrated with the VLC enabled mobile device 105 may also be used to capture barcode data.

The data downloaded to the VLC enabled mobile device 105 is then used to securely transfer the data to a VLC enabled device 109 in an operating room (OR) via point to point VLC 107. For example, a user may receive data for a particular day or days through a wireless download from the data storage system 101. The user may then use the data to aid in the medical procedure in the OR and additionally transfer data to other VLC enabled devices in the OR. The data is transferred by targeting the light transmitting portion of the mobile device 105 in the line of sight of a light detecting sensor of the OR VLC enabled device 109. In one embodiment, the point to point light communication occurs from a user choosing what data to send to the receiving device by simply selecting and/or accepting the data to be transferred on the mobile device 105. In another embodiment, the point to point light communication occurs when the data is transferred when a primer signal such as a key, or an encrypted code is accepted, matches, and/or can be decrypted between the VLC enabled mobile device 105 and the VLC enabled OR device 109. Therefore, only the correct data is transferred to the appropriate OR device 109.

In one embodiment, the surgical plan data may be separated into packets. Each packet consisting of a primer signal, the surgical plan data, and a terminating signal. Each operating room VLC enabled device 109 may be programmed to read a specific primer signal or key. Thereby, when the VLC enabled mobile device is in the line of sight of the VLC enabled OR device, the primer signals are sent until the correct primer signal is read by the OR device. The corresponding surgical plan data is then transferred to the correct OR device 109 until the terminating signal is read. In this capacity, the mobile device 105 may download different types of surgical plan data for different OR VLC enabled devices 109 and the data is targeted to those specific devices.

The VLC enabled mobile device 105 works as a secure way to transfer data in and out of an operating room. For example, each surgical plan may include an equipment check list to build a surgical tray for a particular patient operation or case. The check list of any required equipment (e.g. cutters, motors, drills, a particular model of a robotic surgical system) may be displayed on the VLC enabled mobile device 105 for a specific surgical plan. The contents of a tray may be verified by manually check marking, on the display, that the equipment is present or via an optical image, barcode scanner, or radiofrequency identification (RFID) tag that is read by the mobile device 105 for a set of barcoded or RFID readable equipment . . . . The surgical plan on the mobile device 105 may also include and display patient identification information, the patient's operating site/anatomy, and other general information, so the patient can be verified in the OR at the start of or prior to the surgery by scanning or manually verifying their identification tag. The displayed patient information and verification step may be used prior to uploading surgical plan data to ensure the correct plan is uploaded to any OR VLC enabled equipment 109. In particular, before a surgical plan can be uploaded to an OR VLC enabled device 109 (e.g. a robotic surgical system), both the patient information and a completed equipment check list should be checked and confirmed. In a specific embodiment, the plan or plans may synchronize with the hospital case management scheduling system and notify the user via a standard mobile device when and/or in which OR a surgical procedure is about to start.

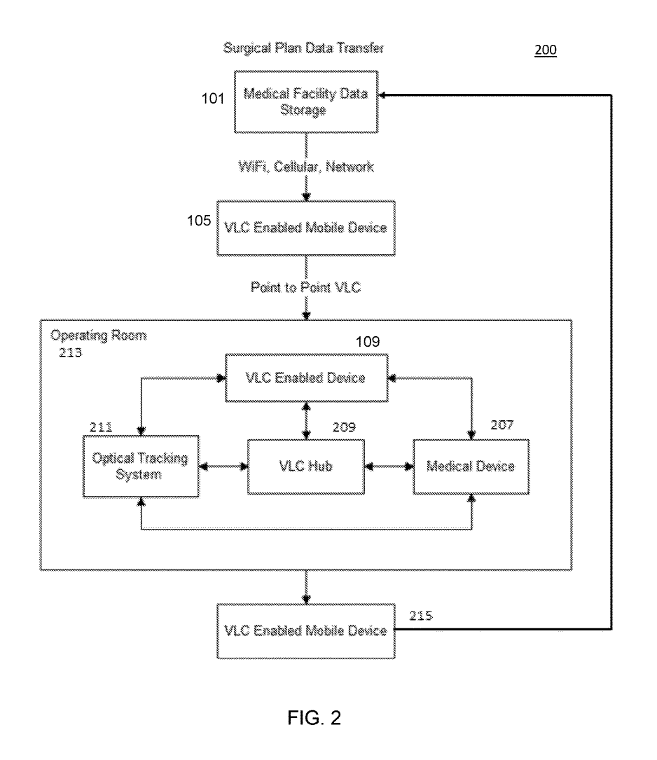

The operating room may contain specific equipment to perform a surgical procedure or procedure/s. With reference to FIG. 2 of a system 200, the equipment in the OR may include an optical tracking system 211, possibly permanently mounted in the OR, for example onto or inside a surgical lamp head, and that this equipment could link to other OR equipment such as tracked markers, medical devices 207 like a surgical robot, or other VLC enabled devices 109 that may receive data embedded in the transferred plan, the plan itself, or information derived from the surgical activities in conjunction with the plan. In one embodiment, the VLC enabled mobile device 105 may transfer data via VLC to one piece of equipment in the OR, and that piece of equipment may subsequently transfer data to other equipment by other electrical or wireless systems. In another embodiment, the VLC enabled mobile device 105 may transfer different data to different equipment via VLC. For example, the VLC enabled mobile device 105 may transfer data about the geometry of the tracked markers to the optical tracking system 211 and then send a pre-operative surgical plan to a medical device 207. The user may place the transmitting portion of the mobile device 105 in the line of sight of the desired VLC enabled OR device receiver 109 and select what data to send. In another embodiment, the data can be similarly transferred by the methods explained above with a primer signal such as a key or encrypted code.

The equipment in the OR may likewise be in communication with the VLC enabled mobile device 105. For example, the mobile device's screen and user interface may be used for setting/reading the status of the devices in the OR via VLC such as the equipment's battery life, calibration date, number of uses, or any required maintenance. In one embodiment, this data from the equipment can be uploaded back the data management system 101 so another department can view or be informed of this data. For example, the data management system may notify the maintenance department that the OR equipment requires maintenance, to replace the battery, calibration status or notify them of an upcoming calibration date.

In specific inventive embodiments, a computer-assisted surgical system is present in the medical care facility. The computer-assisted surgical system includes a planning workstation for creating surgical plans to be executed by an OR computer-assisted surgical device. The computer-assisted surgical system may be for example the TSolution One™ Surgical System (Think Surgical, Inc., Fremont, Calif.) or the RIO® Interactive Orthopedic System (Stryker Mako, Ft. Lauderdale, Fla.). The computer-assisted surgical system is often operated by different user groups, a user group being a particular surgeon and their medical care team. To ensure the correct surgical plan is associated with the correct user group, internal information (e.g. surgeon name, group ID/usernames, passwords) for the user group/team may be created on the VLC enabled device 105 at the planning workstation. Therefore, any data or surgical plan(s) created on the planning workstation can only be accessed or transferred by inputting the correct internal information. This provides additional security and reduces the possibility of transferring incorrect data in the OR for a particular procedure. In addition, battery chargers for the VLC enabled device 105 can be located at a docking station at the planning workstation or the base of the computer-assisted surgical device for the duration of planning or the duration of a procedure.

In a particular embodiment, in order to execute a computer-assisted surgical procedure, the patient's operative bone needs to be registered so the coordinates of the bone is known with respect to the computer-assisted surgical device. Traditional methods of registration are described in U.S. Pat. Nos. 6,033,415 and 8,287,522. The VLC enabled mobile device 105 may be adapted into a digitizer to aid in the registration process. For example, a probe of known geometry may be attached to the VLC enabled mobile device 105. The display may guide the process of registration by displaying the operative bone and the location of the points to be collected on the bone. Tracking markers (e.g. optical markers, radiofrequency transmitters, special characters or symbols) may be attached to the VLC enabled mobile device 105 to track the position and orientation of the probe tip with a tracking system. In a specific embodiment, the tracking markers are displayed directly on the display of the VLC enabled mobile device 105.

Each computer-assisted surgical device may also contain a unique surgical device identification tag, such as a barcode containing the model and serial number of the device. The VLC enabled mobile device 105 may scan the surgical device identification tag prior to uploading a surgical plan to confirm the correct surgical device is used. The surgical device identification information can be uploaded to the data management system 101 to trace the use of the surgical device. Additionally, prior to uploading a surgical plan to a particular surgical device, the previous plan should already be finished. If a user scans the surgical device identification tag to upload a new plan, and the previous plan is still not finished, a prompt may be displayed asking the user for confirmation to reset the robot.

The computer-assisted surgical device also often requires the use of disposable and re-usable tools. A cutter or burr used to mill the bone is a disposable, which is discarded after ever surgical procedure. A motor operating the burr or mill may be used more than once, up to a maximum number of uses prior to replacement. In a particular embodiment, when the barcode a re-usable tool is recorded, the number of uses of the tool can be updated in the planning workstation when the VLC enabled mobile device is returned to the planning workstation. For the next procedure, when the tool is scanned again, if the maximum number of uses for the tool is reached, the check list and verification procedure described above will not allow the tool to pass. Since the equipment/tool check list verification process did not pass, the surgical plan cannot be transferred to the surgical device. This protects against the use of non-conforming tools and equipment, which decreases the chance of medical device malfunctions.

In another embodiment, the operating room is equipped with a VLC hub 209 defined via a collection of OR equipment, where the mobile device 105 acts as the secure way to bring data into or out of the OR from this inter-OR system. The VLC hub 209 consisting of a light detecting sensor, a processor in communication with the sensor to process and read the data and a transmitter to send data to other equipment or back to the VLC enabled device 109. The VLC enabled mobile device 105 may transfer data to the VLC hub 209, which may be in communication with other equipment in the OR such as a medical device 207, optical tracking system 211, or other VLC enabled devices 109. The VLC enabled OR hub 209 may additionally be in communication with a monitor that may display surgical plan data such as patient information, the surgical procedure, surgical plans, the progress of a procedure, or the operating status of other devices in the operating room. In one embodiment the VLC hub 209 may be in communication with other devices by electrical or wireless systems. In another specific embodiment the VLC hub 209 may be in communication with the other devices (109, 207, 211) via VLC communication. For example, the VLC hub 209 may communicate with a VLC optical tracking system 211 that may be used to locate, track, or navigate other devices in the OR such as a medical device 207. Similarly, the VLC hub 209 may be in VLC communication with a VLC medical device 207 to transfer surgical plan data, such as a pre-operative surgical plan to a surgical robot. In one embodiment, the VLC enabled mobile device 105 may be used for updating the software/firmware of VLC equipped devices in the OR, through the VLC hub 209, or directly to the VLC equipped devices.

The VLC hub 209 may also be used as a centralized way of collecting information from the OR equipment. All of the existing devices may transmit their status, including battery life, calibration date, number of uses, any required maintenance to the VLC hub 209 and/or the VLC enabled mobile device 105 to be shown on the screen of the VLC enabled device 109 or a monitor in the OR in communication with the VLC hub 209. This status information may be uploaded to the data management system 101 which can send information and updates to the maintenance department or a medical device manufacturer.

In a specific inventive embodiment, the VLC enabled mobile device 105 downloads software executables that may be uploaded and run with the OR equipment for an arbitrary surgical procedure, with or without the use of an associated surgical plan. For example, a multi-functional medical device may be capable of performing different medical procedures for a specific medical application and/or similar medical procedures for different medical applications. Therefore, one entity may develop an application or software executable that can be used with another entities OR equipment, whereby the VLC enabled mobile device 105 and/or VLC hub 209 may be used to upload these executables, applications, or executable versions to the equipment. The software executables may or may not be associated with surgical plan data. The OR equipment (207, 211) may have a validation process to accept and/or retain the uploaded software executables, application executable or executable versions to comply with regulatory standards and ensure the procedure executes safely and as intended.

In a specific inventive embodiment, the data management system 101 may be used to cache application executable versions, where the download to the VLC enabled mobile device 105 only occurs if the product/version indicated for the plan is not already present on the VLC enabled mobile device 105. Likewise, the OR equipment (207, 211) may cache application executable versions, where the download to the OR equipment (207, 211) from the VLC enabled mobile device 105 and/or VLC hub 209 only occurs if the product/version indicated for the plan is not already present on the OR equipment (207, 211). This may be used as an additional point to point visible light communication technique whereby data is sent only if the cached version is not already present ensuring only the new executables and/or plans are transferred to the OR equipment (207, 211).

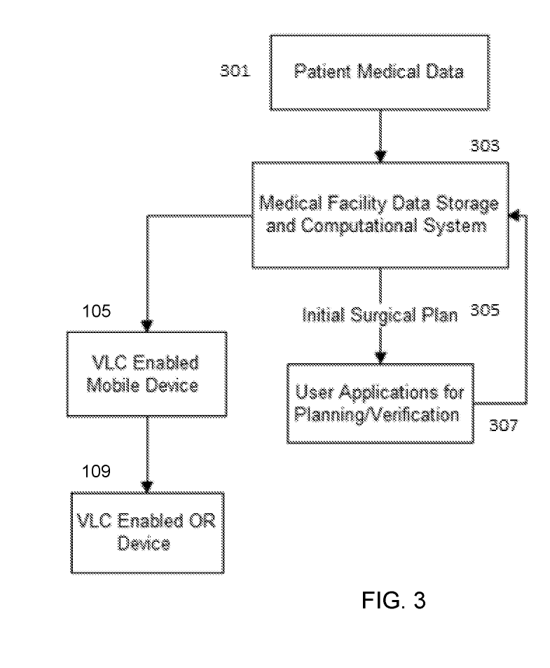

In the embodiment as shown in FIG. 3, the data storage system 101 may be part of a larger cloud based data management and computational system 300 which may include patient medical scan data uploads 301 wherein the computational system 303 processes the medical scan data to generate initial surgical plans 305. The initial surgical plans 305 may be processed and confirmed by technicians and/or surgeons through a set of cloud based SaaS applications, desktop or tablet applications 307, which, when finalized is available for download to the VLC enabled mobile device 105. The plan can then be transferred into the OR directly to a VLC enabled OR device 109 such as a medical device 207 (e.g. a surgical robot, or an optical tracking system), or the OR VLC hub 209. Therefore, the pre-operative surgical plan for the medical device can be quickly verified within minutes of a procedure and then transferred to the operating room 213, which alleviates the necessity to store and transfer the data using a physical transport medium.

The VLC enabled mobile device 105 may also download via VLC the data collected during a case from the OR equipment. The data may be transferred back into the data storage system 101 represented as 215 in association with the surgical case planning. The download of the case data may be required by the OR equipment for closure of the case prior to starting another case as a way to ensure that this data is returned back to the data storage system 101.

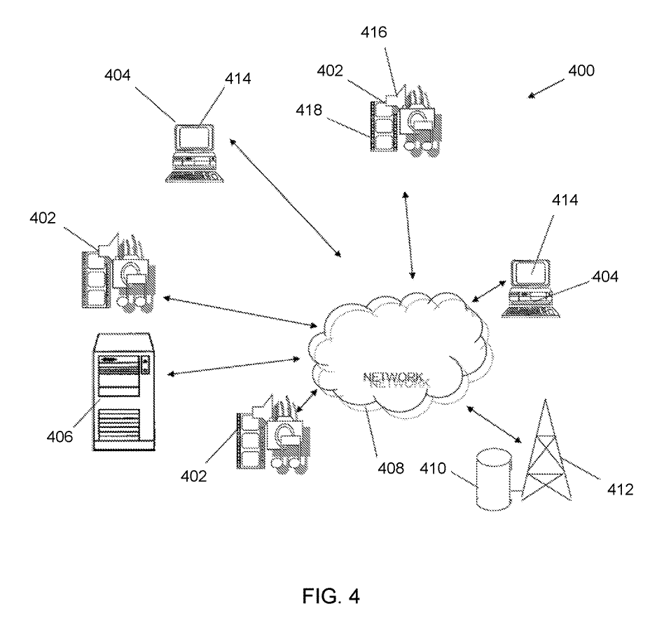

FIG. 4 is a schematic diagram illustrating an overall view of communication devices, computing devices, and mediums for implementing a system and method for using point to point visible light communication for the safe and secure transfer of data in a medical care setting.

The system 400 includes multimedia devices 402 and desktop computer devices 404 configured with display capabilities 414 and processors for executing instructions and commands. The multimedia devices 402 are optionally mobile communication and entertainment devices, such as cellular phones, tablets, and mobile computing devices that in certain embodiments are wirelessly connected to a network 408. The multimedia devices 402 typically have video displays 418 and audio outputs 416. The multimedia devices 402 and desktop computer devices 404 are optionally configured with internal storage, software, and a graphical user interface (GUI) for carrying out elements of the data transfer using point to point visible light communication for the safe and secure transfer of data in a medical care setting according to embodiments of the invention. In a specific embodiment the multimedia devices are configured with VLC transmitters and receivers for interacting with medical devices configured for VLC operation. The network 408 is optionally any type of known network including a fixed wire line network, cable and fiber optics, over the air broadcasts, local area network (LAN), wide area network (WAN), global network (e.g., Internet), or intranet, with data/Internet capabilities as represented by server 406. Communication aspects of the network are represented by cellular base station 410 and antenna 412. In a preferred embodiment, the network 408 is a LAN and each remote device 402 and desktop device 404 executes a user interface application (e.g., Web browser) to contact the server system 406 through the network 408. Alternatively, the remote devices 402 and 404 may be implemented using a device programmed primarily for accessing network 408 such as a remote client.

The software for the apportionment of advertisement display time on digital signage based on a bidding process of embodiments of the invention may be resident on tablets 402, desktop or laptop computers 404, or stored within the server 406 or cellular base station 410 for download to an end user. Server 406 may be implement as a cloud-based service for implementing embodiments of the data transfer method with a multi-tenant database for storage of separate client data for each independent medical facility.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient road map for implementing the exemplary embodiment or exemplary embodiments. It should be understood that various changes can be made in the function and arrangement of elements without departing from the scope as set forth in the appended claims and the legal equivalents thereof.

1. A method to securely transfer data to aid in a medical procedure comprising:
downloading a set of data from a data storage system via a network to a visible light communication (VLC) enabled mobile device;
transferring the set of data from the VLC enabled mobile device to a VLC enabled device located in an operating room (OR) via point to point VLC; and
using the transferred set of data to aid in a medical procedure.

2. The method of claim 1 wherein the VLC enabled device in the OR is a VLC hub in communication with one or more pieces of OR equipment.

3. The method of claim 2 wherein the OR equipment is a surgical robot or an optical tracking system.

4. The method of claim 1 further comprising verifying equipment to be used in the medical procedure with the VLC enabled mobile device.

5. The method of claim 1 further comprising verifying a patient to undergo a procedure with the VLC enabled mobile device.

6. The method of claim 1 wherein said set of transferred data further comprises surgical plan data; and
wherein the method further comprises transferring the surgical plan data and a set of medical procedure data generated during the medical procedure back to the data storage system.

7. The method of claim 6 wherein the surgical plan data further comprises a primer signal for point to point VLC.

8. The method of claim 1 wherein said set of transferred data further comprises a set of software executables for use on operating room (OR) equipment to perform a medical procedure.

9. The method of claim 8 wherein the transferring of the data or software executable only occurs when a data version or an executable version of the transferred data indicated for the procedure is not already present on the receiving VLC enabled device.

10. The method of claim 1 wherein the VLC enabled device in the OR is a surgical robot or optical tracking system.

11. A method to securely transfer operating room (OR) data to a data management system to be accessed in a medical care facility comprising:
transferring data from the OR to a visible light communication (VLC) enabled device via point to point VLC;
uploading the data from the VLC enabled device to a data management system; and
storing the uploaded data in the data management system to be accessed by a medical care facility.

12. The method of claim 11 wherein said uploading of data is through a wireless network.

13. The method of claim 11 wherein said VLC enabled device is a VLC enabled mobile device.

14. The method of claim 11 wherein said VLC enabled device is a VLC hub in the operating room.

15. A method to transfer software executables on operating room (OR) equipment to perform a medical procedure comprising:
downloading a software executable via a network to a visible light communication (VLC) enabled device;
transferring the software executable from the VLC enabled mobile device to the OR equipment via point to point VLC; and
executing the software executable on the OR equipment to perform a medical procedure.

16. The method of claim 15 wherein the transferring of the data or software executable only occurs when the data version or executable version indicated for the procedure is not already present on the receiving VLC enabled device.

17. A system for implementing the method of claim 1.
